(19) United States
(12) Patent Application Publication
(10) Pub. No.: US 2011/0178407 A1
(43) Pub. Date: Jul. 21, 2011

(54) Hard and Soft Backing for Medical Ultrasound Transducer Array

(75) Inventors: Xuan-Ming Lu, Issaquah, WA (US); Gregg W. Frey, Issaquah, WA (US); Richard Morris, Maple Valley, WA (US)

(73) Assignee: Siemens Medical Solutions USA, Inc., Malvern, PA (US)

(21) Appl. No.: 12/690,865

(22) Filed: Jan. 20, 2010

(51) Int. Cl. A61B 8/00

(52) U.S. Cl. 600/459

(57) ABSTRACT

A transducer for multiple purposes is provided. Different backings are used for different elements of a same array. The different backings optimize the respective elements for the desired use. A soft backing (e.g., Z=3 Mrayl) is used behind some elements for ultrasound imaging. A hard backing (e.g., Z=100 MRayl) is used behind other elements for lower frequency operation.

### BACKGROUND

The present invention relates to a transducer for multiple purpose ultrasound. In particular, a transducer is provided for ultrasound imaging at different frequencies and/or for acoustic transmission for other ultrasound purposes.

In addition to imaging, ultrasound may be used to create strain through remote palpitation, create streaming of fluids within masses or cysts, create shear waves, move contrast agents or microbubbles, break or destroy microbubbles, heat tissue, force detectable movement of tissue, or other purposes. Designing an ultrasound transducer for imaging at one frequency as well as other purposes may be difficult. The other purposes may demand greater transmit power at one frequency, and the imaging may demand a wide bandwidth at another frequency. For example, 1 to 2 MHz frequency for one purpose and a 4-5 MHz or higher frequency for an imaging purpose are desired. A transducer with limited bandwidth may not operate efficiently for both applications.

### BRIEF SUMMARY

By way of introduction, the preferred embodiments described below include multi-purpose transducers and methods for using a transducer for multiple purposes. Different backings are used for different elements of a same array. The different backings optimize the respective elements for the desired use. For example, a soft backing is used behind some elements for ultrasound imaging. A hard backing is used behind other elements for lower frequency operation.

In a first aspect, an ultrasound transducer is provided for multi-purpose use. Transducer elements of an array are operable separably for transducing between acoustic and electrical energies. The array has a face at which the acoustic energies are transmitted and received and a back opposite the face. A first backing is adjacent the back of the array. The first backing is adjacent a first sub-set of the elements. A second backing is adjacent the back of the array. The second backing is adjacent a second sub-set of the elements where the second sub-set different from the first sub-set. The first backing is a hard backing material. The hard backing material has an acoustic impedance greater than the transducer layer, such as greater than PZT5H, PMN-PT single crystal. The second backing is a soft backing material. The soft backing material has an acoustic impedance less than the transducer layer.

In a second aspect, method for multi-purpose use of a medical ultrasound transducer is provided. Acoustic energy is transmitted at a first frequency from a first plurality of first elements. The first elements have a ¼ wavelength resonance at the first frequency. The ¼ wavelength resonance is promoted by a backing with an acoustic impedance greater than a transducer material of the first elements. Acoustic energy is transmitted at a second frequency from a second plurality of second elements. The second elements have a ½ resonance at the second frequency. The ½ resonance is promoted by a backing with an acoustic impedance less than the transducer material of the second elements. The first and second elements are part of a same array.

In a third aspect, an ultrasound transducer is provided for multi-purpose use. First elements are configured for transmission at a first frequency by a first backing. Second elements are configured for transmission at a second frequency, higher than the first frequency, by a second backing. The second backing is softer than the first backing. The first and second elements are adjacent each other in an array.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is an example illustration of two adjacent elements of an array with different backing.

FIG. 2 is an example illustration of a layered backing;

FIG. 3 is an example illustration of reflection for the three layer embodiment of backing shown in FIG. 2;

FIG. 4 illustrates PZT resonance modes for hard and soft backing respectively;

FIG. 5 shows two way response High Frequency (HF) and Low Frequency (LF) array as a function of frequency for elements with hard and soft backing;

FIG. 6 shows an example of transducer element electric impedance for hard backing and soft backing for a same center frequency.

FIG. 7 is cross-section and bottom view of an array with different backings according to one embodiment.

FIGS. 8, 9 and 10 are bottom views of arrays with different backing patters according to other embodiments;

FIG. 11 shows one embodiment of a method for multi-purpose use of a medical ultrasound transducer.

### DETAILED DESCRIPTION OF THE DRAWINGS AND PRESENTLY PREFERRED EMBODIMENTS

Ultrasound transducers and associated connections with an ultrasound system are provided for multiple purpose ultrasound. A dual frequency transducer can be made with the same PZT but different (e.g., soft and hard) backing for different elements. For example, the ultrasound transducer has low frequency (LF) elements for radiation force or therapeutic ultrasound, and high frequency (HF) elements for imaging used by acoustic force radiation imaging (ARFI), high intensity focused ultrasound (HIFU), contrast imaging, harmonic imaging, or similar application. Other combinations, such as imaging at different frequencies or transmit at one frequency and receive at another frequency, may be used. Such dual frequency transducer can be made with the same PZT thickness but backings with substantial impedance differences.

Using a hard backing for some elements may have other advantages. Hard backing (e.g., tungsten carbide) may provide high thermal conductivity.

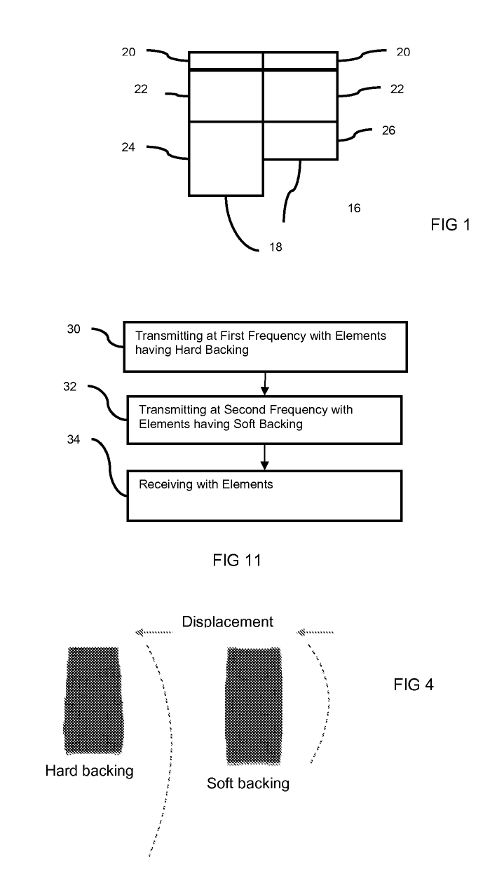

FIG. 1 shows one embodiment of part of an array 16 of elements 18 for multi-purpose use. The array 16 is part of a one-dimensional (1D), or multidimensional transducer (e.g., 1.25D, 1.5D, 1.75D, or 2D). The array 16 is within a transducer probe, such as a handheld probe, catheter, intra-cavity, or other probe. Only two elements 18 are shown for simplicity, but the array 16 may include additional elements 18, such as tens, hundreds or thousands of elements 18.

The array 16 has a face at which the acoustic energies are transmitted and received. The face is flat, concave or convex. The face is adjacent an acoustic window of the probe and/or is placed adjacent a region to be scanned. Opposite the face of the array 16 is a back. The back is away from the acoustic window, such as being further away from the acoustic window. The back of the array 16 is positioned adjacent to backing 24, 26, such as the bottom of the transducer layer 22 as shown being a back of the array 16.

Each transducer element 18 of the array 16 includes one or more transducer layers 22, one or more matching layers 20, one or more backing layers 24, 26, electrodes, cables connected with respective electrodes, and tuning circuits. Additional, different or fewer components may be provided. For example, a switch or multiplexer is provided for connecting a same cable or a same transmit channel to different ones of the elements 18 and associated electrodes. The switch or multiplexer may be provided between the tuning circuits and the transmitter or along the cable where similar frequencies are to be used for the different purposes. The tuning circuits and cables are provided outside the probe. Alternatively, the tuning circuits are within the probe.

Each element 18 is operable for transducing between acoustic and electrical energies. The transducer layer 22 converts electrical energy to acoustic energy for transmission. Most of the generated acoustic energy propagates towards the face and back of the array 16. The transducer layer 22 converts received acoustic energy into electrical energy. The electrodes are provided on opposite sides of the transducer layer 22 for operation of the transducer.

The elements 18 are acoustically and electrically isolated for separate operation. Kerfs, epoxy, or other spacing between elements 18 may provide acoustic isolation. A bridge or common transducer material may alternatively be connected between the elements 18. Separate electrodes may provide electrical isolation. A common ground electrode may be shared by some or all of the elements 18. Multiple elements 18 may share a transmit or receive electrode, such as in a matrix system or a system forming macro elements.

The one or more transducer layers 22 are formed from a same or different transducer material. For example, a single crystal, such as PZN-PT or PMN-PT, is used. Other piezoelectric materials, such as PZT5H, or composite materials may be used. In another embodiment, the transducer layer 22 is formed from a semiconductor material as a microelectromechanical or CMUT device. In yet another embodiment, one or more of the transducer layers 26, 28 are formed from an electrostrictive polymer.

In one embodiment, all of the elements 18 are formed from a same block of transducer material. The transducer layer 22 of each element 18 is a same thickness, number of layers, and material, such as forming the transducer layer 22 for each element 18 by kerfing a block of PZT. The same thickness of the transducer layer 22 between the emitting face and the back is provided. In other embodiments, different elements 18 have different structures, such as a different number of transducer layers 22, different thickness, and/or different materials.

The acoustic impedance of the transducer layer 22 of each element is between 10 and 50 MRayl. Piezoelectric ceramic has an acoustic impedance of about 30 MRayl. Different material mixtures or formation processes may result in deviation from 30 MRayl. About accounts for this deviation as well as due to the addition of multiple layers of transducer material or other material. Composite transducer layers 22 (e.g., posts or slabs of piezoelectric material held together or surrounded by epoxy) may have a different acoustic impedance.

The elements 18 are optimized for different purposes. For example, one element 18 or sub-set of elements 18 of the array 16 is optimized for imaging or transmitting and receiving acoustic energy. Another element 18 or sub-set of elements 18 of the array 16 is optimized for another purpose. Different purposes include only transmission and not reception, or transmission of higher power. In another embodiment, the elements 18 are optimized for different frequency characteristics even if both types of elements 18 are used for transmission and/or reception. For example, one type of elements 18 in the array 16 has a wider bandwidth or different frequency response than another type of elements 18 in the array 16. As another example, the different elements 18 are optimized for use at different center frequencies.

The different types of elements 18 are mutually exclusive. Where the physical structure of the element 18, such as the backing 24, 26, distinguishes between the type of element 18, the transducer elements 18 of one type are exclusive to that type. Some elements 18 are of one type and other elements 18 are of another type or types. Each sub-set is exclusive. In alternative embodiments, the element 18 may have switchable characteristics, so may be programmed or controlled to be a selected type.

Different types of elements 18 are used for different purposes. For example, one sub-set of elements 18 is used for imaging, and the other sub-set of elements 18 is used for pushing tissue, fluid or microbubbles, for breaking microbubbles or tissue structures, or for heating or other therapy. The different uses may dictate different design or optimization. For example, higher frequency imparts greater radiation force. For tissue palpitation or other uses, one type of element 18 is adapted for higher frequency than used for imaging. As another example, microbubbles are more reactive to lower frequencies in some cases. Greater movement or even destruction may be provided with lower frequencies than used for imaging. Bandwidth or ability to operate pursuant to different amplitude levels may alternatively or additionally be used.

Different ones of the elements 18 may be used for different purposes at different times, such as using one type for imaging in one circumstance and the other type for imaging in another circumstance. For example, one type may used for CW or color Doppler mode, while another type is used for B-mode. Similarly, different elements 18 may be used for non-imaging purposes at different times, such as using one type adapted for higher frequency for imaging at one time but remote palpitation at other times. Multiple types of elements 18 may be used for a common purpose. For example, both types are used to generate acoustic energy for non-imaging purposes. More than two types may be provided, such as providing a third type for implementing yet another purpose or for use with either of the other types of elements 18 for imaging or other purpose.

In addition to any difference in the transducer layers 22 or to optimize the elements 18 differently even with the same transducer layer 22, the backings 24, 26 of some of the elements 18 are different from other elements 18. The backings 24, 26 may promote operation or provide configuration of the elements 18 for different frequencies. For example, different elements 18 are used for transmitting at different frequencies. The backings 24, 26 are optimized or designed for operation at the different frequencies.

The backing layers 24, 26 are different by number of layers, type of material, acoustic absorbance, composition, shape, size, depth, other characteristic or combinations thereof. In one embodiment, the backing layers 24, 26 have different material with different corresponding amounts of hardness. One backing layer 24 is softer than another backing layer 26. The difference in hardness may result in a difference in acoustic impedance of the backing layers 24, 26, such as one backing layer 26 having an impedance above the acoustic impedance of the transducer layer 22 and another backing layer 24 having an impedance below the acoustic impedance of the transducer layer 22.

The backing layers 24, 26 are adjacent to the back of the array 16, such as being adjacent to the bottom (as shown in FIG. 1) of the transducer layer 22. An electrode, such as metalized flexible circuit material, or other layers may be positioned between the transducer layer 22 and the backing layers 24, 26.

In the example of FIG. 1, the hard backing layer 26 is used for some of the elements 18 of the array 16 and not for others. This backing layer 26 is formed from hard backing material. A hard backing material has a greater acoustic impedance than the material of the transducer layer 22, such as having an acoustic impedance of at least 50 MRayl, 60 MRayl or even higher. For example, the acoustic impedance of the backing is 2-3 times or more than the acoustic impedance of the transducer layer 22. This greater acoustic impedance may better cause the element 18 to operate in a ¼ wavelength resonant mode. The hard backing may have a lesser acoustic impedance or hardness, but still greater than the soft backing of the soft backing layer 24.

The hard backing layer 26 is configured for operation at a desired frequency by thickness along the acoustic propagation dimension. For example, the thickness is selected to be ¼ wavelength of a desired center frequency. For a 3 MHz center frequency, the thickness is about 300 microns, which would be about 600 microns if the soft backer is used. The thickness and acoustic impedance promote resonation of the element 18 at the desired frequency. This resonation may provide a stronger electric field or a greater acoustic output from the emitting face for a same voltage in the hard backing case. A stronger electric field or greater acoustic output allows for operation with a lesser voltage. Other thicknesses may be used, including constant or variable thickness.

The hard backing may allow reduction in thickness of the transducer layer 22 as compared to conventional soft backing and may allow reduction of the elements electric impedance, which is proportional to the transducer layer thickness. The element impedance depends on frequency, element size, and type of piezoelectric material. The transducer elements have a range of a few hundreds to tens of kilo-Ohms impedance typically. Reducing the element impedance improves electric impedance matching to cable impedance (50 to 100 Ohms), which may help the system to deliver more power to the elements.

The hard backing (HB) (e.g., Z>60MRayl where Z is acoustic impedance) may behave as a “fixed” boundary condition, so may promote PZT resonation at ¼ wavelength. A soft backing (SB) (e.g., Z˜3MR) may act as “free” boundary condition, so may promote PZT resonation at ½ wavelength. For the same PZT thickness, the resonance frequency with the hard backing (HB) is about half of the soft backing, which may be used for low frequency operation (e.g., 2-3 MHz) while the soft backing array may be used for higher frequency operation (e.g., 5-6 MHz).

FIG. 4 illustrates PZT resonance modes for hard and soft backing, respectively. The hard backing forms a more rigid surface, causing different deformation of the transducer layer as compared to transducer material backed with the less rigid surface of the soft backing. The displacement of the transducer material is different depending on the hardness of the backing.

The backing layer 26 of hard backing material is a composite or solid material. For example, the backing layer 26 is formed from a solid. The backing layer 26 is entirely formed as one structure or material. Solids with the desired acoustic impedance or hardness may be used, such as tungsten (about 100 MRayl), tungsten carbide (about 60-101 MRayl), or uranium (about 60 MRayl). Various metals, ceramics, cemented carbides, glass, or other materials may be used.

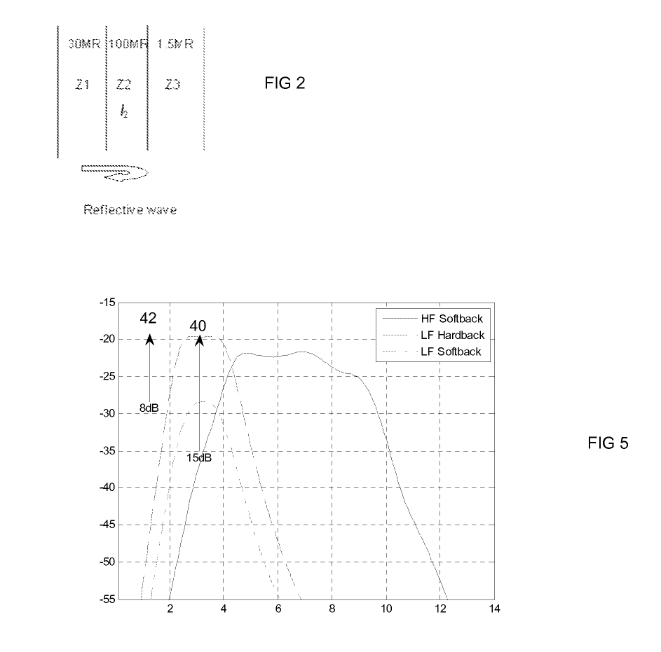

In other embodiments, the backing layer 26 includes a plurality of different materials. The different materials may be mixed, such as a composite backing. The different materials may be stacked or layered, such as associated with forming an anechoic surface backing. In one embodiment shown in FIG. 2, different materials are stacked or layered along an acoustic propagation dimension (i.e., layered generally parallel with the emitting face of the array 16). FIG. 2 shows the transducer layer Z1 as having 30 MRayl acoustic impedance. The next layer Z2 is the hard backing material having a 100 MRayl acoustic impedance. The final or next layer Z3 is a soft backing material (e.g., RTV) having a 1.5 MRayl acoustic impedance. Other layers may be provided. The additional soft backing Z3 is the same or different from the soft backing used for elements 18 without the hard backing of layer Z2. For example, the soft backing is cast onto the array after bonding the hard backing to the array. The soft backing is cast over all of the elements 18 while only some of the elements 18 include the bonded hard backing.

The additional soft backing provides a boundary with a substantial acoustic impedance difference. The difference causes greater reflection of any impinging acoustic energy. The reflections may be at any frequency, such as frequencies for which the elements 18 with the hard backing layer 26 are used. For example, the difference may cause reflection greater than 95% of an impinging acoustic wave. The reflection may minimize backing thickness and heat generation.

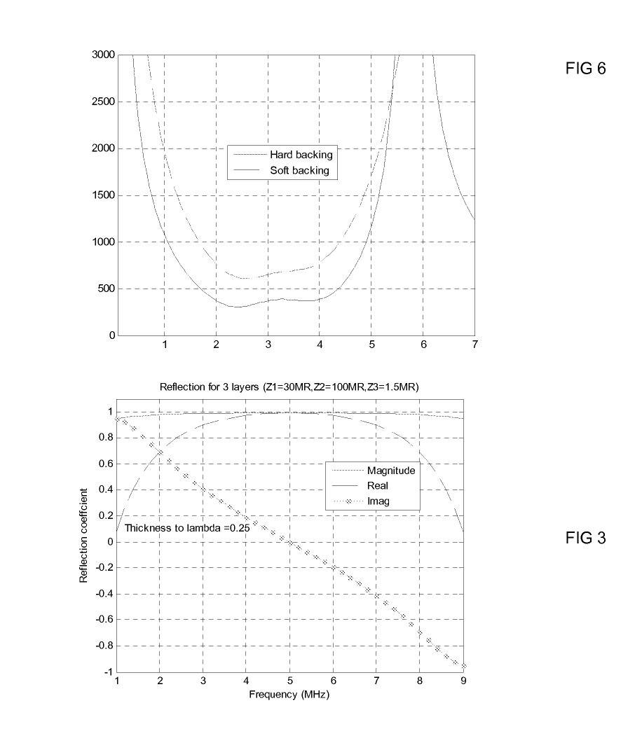

The reflection coefficient of the three layers shown in FIG. 2 may be calculated as:

$$R = \frac{( 1 - \frac{{z}_{1}}{{z}_{2}} ) cos ( {k}_{2} {l}_{2} ) + j ( \frac{{z}_{2}}{{z}_{3}} - \frac{{z}_{1}}{{z}_{2}} ) sin ( {k}_{2} {l}_{2} )}{( 1 + \frac{{z}_{1}}{{z}_{2}} ) cos ( {k}_{2} {l}_{2} ) + j ( \frac{{z}_{2}}{{z}_{3}} + \frac{{z}_{1}}{{z}_{2}} ) sin ( {k}_{2} {l}_{2} )}$$

where k₂ is the wave number and is equal to ω/c (ω is the angular frequency and c is the speed of sound). l₂ is the thickness of the hard backing, such as 300 microns. For this example, R is about 0.99 when the thickness is near the quarter wavelength. About 99% of the acoustic energy is reflected. The reflection is maximized with a very high Z layer followed by a very low Z layer. This may reduce the backing thickness or reduce heating in the backing since high impedance backing is often more thermally conductive (e.g. tungsten Carbide thermal conductivity vs. epoxy thermal conductivity). FIG. 3 shows the reflection coefficient for the example of FIG. 2.

In the example of FIG. 1, the soft backing layer 24 is used for some of the elements 18 of the array 16 without any hard backing layer 26. The soft backing layer 24 may or may not be used on all of the elements 18. This soft backing layer 24 is formed from soft backing material. A soft backing material has a lesser acoustic impedance than the material of the transducer layer 22, such as having an acoustic impedance of at most 10 MRayl. For example, the acoustic impedance of the backing is less than the acoustic impedance of the transducer layer 22 by fraction of half or less. This lesser acoustic impedance may better cause the element 18 to operate in a ½ wavelength mode. In other embodiments, the soft backing material matches the acoustic impedance of the transducer layer 22. The soft backing layer 24 may have a greater acoustic impedance or hardness, but still less than the hard backing material of the hard backing layer 26.

The soft backing layer 24 is configured for operation at a desired frequency by thickness along the acoustic propagation dimension. For example, the thickness is selected to be ½ wavelength of a desired center frequency (e.g., PZT layer with thickness of 300 microns resonate at about 5 MHz of center frequency). The soft back layer 24 is configured as a conventional backing layer for medical diagnostic ultrasound imaging transducers, but may have other configurations. The thickness and acoustic impedance promote resonation of the element 18 at the desired frequency. Other thicknesses may be used, including constant or variable thickness.

The backing layer 24 of soft backing material is a composite or solid material. For example, the soft backing layer 24 is entirely formed as one structure or material. As another example, the soft backing layer 24 is formed from a composite or a plurality of different materials. Composites with the desired acoustic impedance or hardness may be used. For example, epoxy, RTV, silicone, or other soft material are filled or mixed with tungsten or other particles to provide the desired acoustic impedance. In one embodiment, epoxy is mixed with 10-20% by volume of tungsten powder. Various plastics, organic composites, polymers filled with graphites, metals, ceramics, glass or other materials may be used. Layers of graduated acoustic impedance or a uniform mix may be provided. For layers, an anechoic surface may be provided. The soft backing layer 24 may be arranged to reduce reflection, instead absorbing acoustic energy. In other embodiments, solids are used for the soft backing layer 24.

The elements 18 with the soft backing layer 24 may be configured for operation at a different or same frequency as the elements 18 with the hard backing layer 26. For example, the hard backing layer 26 is sized for operation at 2-4 MHz, and the soft backing layer 24 is sized for operation at 4-9 MHz. The hard backing layer 26 may be used for lower frequencies (LF) to provide the desired higher peak pressure for pushing or therapy. Examples using soft and hard backing elements 18 are provided.

FIG. 5 shows an example two-way response of hard backing and soft backing comparison. The two way sensitivity for elements 18 with the hard backing may increase ˜15 dB as compared to elements 18 with soft backing at 3 MHz for same PZT thickness (e.g., 300 μm). The two-way sensitivity of an element 18 with hard backing may be improved 8 dB at low frequencies (e.g. 3 MHz) as compared to elements 18 with the soft backing layer 24 with about less than double of the PZT thickness (e.g., 550 μm). The arrow 40 shows the gain difference between hard and soft backing at about 3 MHz for the same PZT thickness. The arrow 42 shows the gain difference between using the soft backing element with two layers of transducer material for pushing at low frequency as compared to using the hard backing element with a single layer of transducer material. The imaging array with soft backing is for high frequency operation, such as using soft backing elements 18. The pushing arrays are for ARFI pushing pulses at lower frequencies.

FIG. 6 shows the impedance amplitude as a function of frequency for the hard backing (Z˜100 MRayl) and soft backing (Z˜4 MRayl) for the same center frequency (˜3MRayl). The element impedance reduced almost half for the hard backing compared to soft backing. The lower element impedance often matches better to cable and system impedance.

Two types of elements 18 are shown in FIG. 1, hard and soft backing elements 18. In other embodiments, more than two types of elements 18 are provided. Any differences in elements 18 may be provided, such as each type of element 18 having a different backing arrangement or material.

The different types of elements 18 may be arranged in any desired pattern across the array 16. For example, the hard backing elements 18 are used for low frequency operation and the soft backing elements 18 are used for high frequency operation. The low and high frequency elements 18 may be patterned by distribution of backing layers 24, 26 in any mix and/or ratio to achieve desired performance. The different types of elements 18 are adjacent to each other within the array 16, but some elements 18 of a given type may be surrounded by the same type of elements 18.

In a one-dimensional array, more than two elements 18 may be provided, such as 64, 128, 196 or other numbers of elements 18 in a linear, curved linear, or phased array. The elements 18 are arranged as every other element 18 or every other group of elements 18. As another example, groups of outer or end elements 18 of the array on one or both ends have hard backing layers 26 and other elements 18 (e.g., center elements 18) have soft backing layers 24.

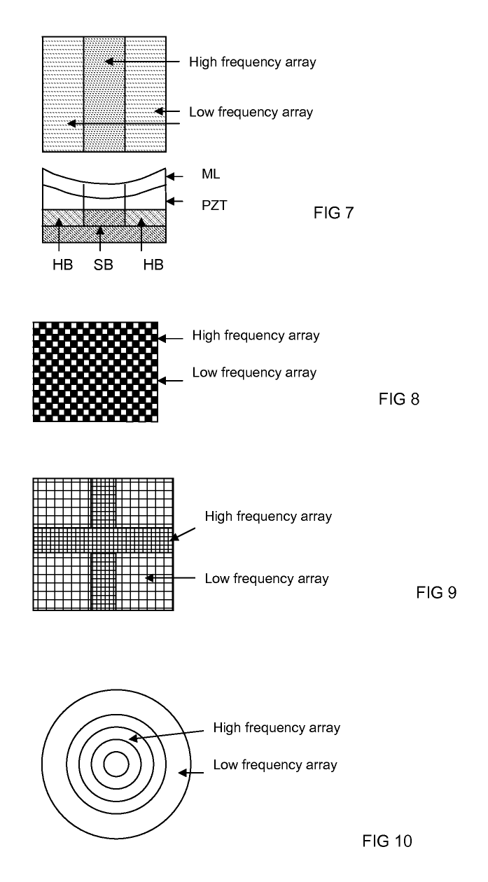

In other embodiments, the elements 18 are part of a multi-dimensional array, such as a 1.25D, 1.5D, 1.75D, or 2D array. Various arrangements of the different types of elements 18 are possible. FIG. 7 shows a 1.25D, 1.5D, or 1.75D arrangement using three rows. Only two or more than three rows may be provided. As shown in FIG. 7, each row is a contiguous line of elements 18 of a same type. The center row has soft backing elements 18 for use at higher frequencies (e.g., 6 MHz) in imaging. The outer two rows have hard backing elements 18 for use at lower frequencies (e.g., 3 MHz) to push or apply therapy. Other arrangements are possible. The outer rows are connected together to the same channels, such as with a 1.25D or 1.5D array. The outer rows may be separately connected to beamformer channels, such as with a 1.75D array.

Each row may have the same size or different sizes of elements 18. The rows may be the same size or different size of elements 18, number of elements 18, and/or arrangement of elements 18. In other embodiments, one or more of the rows may include more than one type of element 18.

FIGS. 8 and 9 show example arrangements by type of element 18 in two dimensional arrays. FIG. 8 shows an every other element 18 or checkerboard distribution in a two-dimensional array. The checkerboard arrangement may minimize or have less side lobes. The checkerboard pattern may be based on every other group of elements 18, such as 1×N, 2×N, 3×N, 4×N, . . . where N is a positive integer (e.g. N=1, 2, 3, 4, or more). Different size groupings may be used in a same array 16.

FIG. 9 shows another pattern. Soft backing elements 18 form a cross array or two intersecting arrays. As shown, the cross is orthogonal, but arrays at other angles to each other may be used. The other elements 18 have hard backing layers 26, providing four arrays of hard backing elements 18. Other arrangements are possible.

FIG. 10 shows another arrangement of low and high frequency array in annular array. The hard backing and soft back may be arranged in the rings of different radius. One example is the large radius and large size ring of elements with hard backing may be used for low frequency pushing and fine pitch and small size rings with soft backing may be used for high frequency imaging.

In the example shown, the hard backing elements 18 are used for lower frequency operation, and the soft backing elements 18 are used for higher frequency operation. Other differences provided by the different types of elements 18 in addition or as an alternative to frequency of operation may be provided. The elements 18 of FIG. 9 have two different sizes. The larger hard backing elements 18 allow for fewer channel connections since these low frequency elements 18 may be used with less resolution. Other arrays with different sized elements 18 may be provided.

The elements 18 and/or the array 16 are flat or concave. FIG. 7 shows a concave arrangement across the rows of the array 16. As a result, each element 18 of the center row has a concave shape. The elements 18 of the outer rows are also concave or flat with an angle to the emitting face. The shape is provided by the transducer layer 22 with the backing layers 24, 26 being free of the shaping. The PZT and/or matching layers 20 and/or 22 are concave. In other embodiments, the shaping is provided by a curve in the array 16 but with flat elements 18. The concave surface is along the elevation dimension in one embodiment. A parabolic or generally curved concave surface to provide focus and associated low loss urethane lens may increase high frequency sensitivity and bandwidth. High frequency signals excite only the center portion of the elevation aperture, resulting in a narrower beam for imaging resolution. The concave surface may be along the azimuth dimension in other embodiments.

FIG. 11 shows one embodiment of a method for multi-purpose use of a medical ultrasound transducer. For example, the transducer is used to image at different frequencies. As another example, the transducer is used to image and to apply acoustic force radiation or therapy. The method is implemented with a transducer disclosed in FIG. 1 or 7-10 or a different transducer. The acts shown in FIG. 12 may be performed in the same or different order. Additional, different, or fewer acts may be provided.

In act 30, acoustic energy is transmitted at a frequency from a first plurality of elements. The frequency is a center frequency of a narrow or broad band pulse or pulses. In one embodiment, the frequency is a relatively lower frequency than used in act 32, such as being 1-3 MHz. In other embodiments, higher or lower frequencies may be used.

The acoustic energy is transmitted in response to application of an electrical signal to the elements. For example, a piezoelectric ceramic is caused to expand or contract in response to a potential difference caused by variation of the electrical signal relative to a ground. As another example, a flexible membrane is caused to flex inward or outward from a chamber in response to the electrical signal. The electrical energy is transduced to acoustic energy in an ultrasound frequency.

Different or the same electrical signals are applied to each of the elements of the plurality. The plurality of elements is of a same or different type. For example, all elements of the plurality (e.g., all elements of a sub-set of elements) have a hard backing. With a hard backing, the elements may operate at a ¼ wavelength resonance mode at or near the transmitted center frequency. The backing promotes the ¼ wavelength resonance by having a thickness at or near the ¼ wavelength, a hardness causing reflection, and/or an acoustic impedance greater than the transducer layer. The thickness may be about the center frequency by being within the bandwidth of the transmit pulse.

Due to the hardness or acoustic impedance difference, the hard backing reflects at least some acoustic energy. In one embodiment, a substantial portion, such as 50% or more, of the impinging acoustic energy is reflected back to the emitting face of the array. For example, about 50% of the total generated acoustic energy is emitted from the face. About another 50% is emitted from the back of the transducer layer. Of the about 50% at the back, a percentage X is reflected back to the emitting face. X may be any value, but greater values increase the transmit efficiency. In one embodiment, X is 50% or more, such as 95% or 99%. Where X is 30%, then 80% of the total energy is transmitted from the emitting face. By having multiple layers with acoustic impedance differences, more energy may be reflected. For example, 90%, 95% or 99% of the energy from the back is reflected using a hard backing and soft backing layers for the same element.

The transmission of act 30 is for imaging or another purpose. For example, the acoustic energy is transmitted for therapeutic ultrasound. Acoustic energy at a desired power set by amplitude, frequency, aperture size, pulse repetition frequency, and/or number of pulses is transmitted to heat tissue, activate drugs, cause cavitations, or break contrast agents. The contrast agents contain drugs or other chemicals for delivery within a patient. By releasing the drugs through destruction of the contrast agents in specific locations, targeted drug delivery is provided. Contrast agent destruction alone may disrupt clots. Transmission of high intensity focused ultrasound (HIFU) may be used.

In another example, the acoustic energy is transmitted as a pushing pulse of acoustic radiation force imaging (ARFI). The acoustic energy is sufficient to cause movement of the tissue. Strain or other tissue movement is created through remote palpitation. Multiple applications of acoustic radiation in rapid succession cause strain or tissue palpitation. The associated displacement may then be imaged, such as disclosed in U.S. Pat. No. 6,371,912, the disclosure of which is incorporated herein by reference. Acoustic energy for measuring elasticity may be used, such as energy to cause compression or expansion of tissue to then measure tissue response.

In yet another example, the acoustic energy is for imaging. The transmission of act 30 is at a fundamental frequency for reception in act 34 at a higher harmonic frequency. This example may not use the transmission of act 32.

In yet another example, act 30 may be used to create streaming of fluid. For example, U.S. Pat. No. 5,487,387, the disclosure of which is incorporated herein by reference, creates movement of fluids for identifying cysts. The acoustic energy is of sufficient intensity to initiate movement of any fluid located within a target lesion. Imaging is then performed of the target lesion to detect the presence or absence of fluid movement.

In another purpose, the acoustic energy is used to create shear waves, such as disclosed in U.S. Pat. No. 6,764,448, the disclosure of which is incorporated herein by reference. Acoustic energy is transmitted into a tissue in a given direction to provide a virtual extended shear wave. The shear wave generates an extended shear wave that propagates in a direction orthogonal to the original direction. The tissue responsive to the shear waves is imaged.

As yet another example of a different purpose for generating the acoustic energy in act 20, acoustic energy adapted to move contrast agents without breaking, such as through power or frequency selection, assists in the targeting of contrast agents. For example, contrast agents are caused to move adjacent to or perfuse into tissue using acoustic energy. Contrast agents may then be imaged or destroyed for release of drugs or destroyed and imaged to determine wash-in times or perfusion rates.

In act 32, acoustic energy is transmitted at a same or different frequency from a different plurality of elements. The frequency is a center frequency of a narrow or broad band pulse or pulses. In one embodiment, the frequency is a relatively higher frequency than used in act 30, such as being 3-6 MHz. In other embodiments, higher or lower frequencies may be used.

The acoustic energy is transmitted in response to application of an electrical signal to the elements. For example, a piezoelectric ceramic is caused to expand or contract in response to a potential difference caused by variation the electrical signal relative to a ground. As another example, a flexible membrane is caused to flex inward or outward from a chamber in response to the electrical signal. The electrical energy is transduced to acoustic energy at an ultrasound frequency.

Different or the same electrical signals are applied to each of the elements of the plurality. The plurality of elements is of a same or different type. For example, all elements of the plurality (e.g., all elements of a sub-set of elements) have a soft backing. With a soft backing, the elements may operate at a ½ wavelength resonance mode at or near the transmitted center frequency. The backing promotes the ½ wavelength resonance by having a thickness at or near the ½ wavelength, a hardness causing absorption or scattering rather than reflection, and/or an acoustic impedance less than the transducer layer. The thickness may be about the center frequency by being within the bandwidth of the transmit pulse.

The transmission of act 32 is for imaging or another purpose. For example, acoustic energy is transmitted after the transmission of act 30 to receive echoes. The patient is scanned to determine the effects of the transmission of act 30 and/or to align the transmissions of act 30 with the tissue of interest.

The transmissions of acts 30 and 32 are performed with different elements of a same array. For example, the transmission of act 30 uses elements with soft backing, and the transmission of act 32 uses elements with hard backing. One or more of the elements may be used in both transmissions. The different elements are configured for operation at different frequencies and/or different acoustic amplitudes in one embodiment. For different frequencies, the performance of dual frequency array with hard backing and soft backing may improve transmit efficiency (Kpa/V) for high pressure at the low frequency band. A gain of about 15 dB at 3 MHz for the hard backing elements as compared to using an imaging array with soft backing for ARFI pushing pulses is shown in FIG. 5. FIG. 5 also shows a gain of about 8 dB if a low frequency array with conventional single layer of transducer material with hard backing is used as compared to a two transducer layer element with soft backing.

In act 34, the elements used for the transmission of act 32 are used for receiving acoustic echoes. Different transducers may alternatively or additionally receive the echoes. The echo signals are generated in response to the transmission of acoustic energy in act 32. The echoes cause compression or expansion of the transducer layer or flexing of a membrane of the transducer. In response to the acoustic echoes, the transducer transduces to an electrical energy.

The electrical energy generated in response to the echo signals is provided to a receiver or receive beamformer. The receive beamformer generates samples or signals representing given spatial locations of the patient scanned using a plurality of channels and associated elements of the transducer. The transducer element or transducer layer is optimized for imaging use, such as having a thickness, material composition, tuning, connection to transmit and receive beamformer channels, or other characteristic for ultrasound imaging.

Reception of echoes is not performed in response to the transmission of act 30. The transmissions of act 30 are for other purposes. Alternatively, the reception of act 34 is performed in response to act 30 as well as act 32. Transmit and reception are provided in sequence, such as transmit in act 30, receive in act 34, transmit in act 32, and receive in act 34. In other embodiments, act 30 is performed and responsive echoes are received in act 34. Act 32 is not performed.

The received echoes may be used to form an image of tissue and/or fluid. The displayed image may be a B-mode, M-mode, color flow, tissue velocity, Doppler mode, spectral Doppler or any other type of image. For example, the image is an elasticity or strain image.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

fch I (we) claim:

1. An ultrasound transducer for multi-purpose use, the transducer comprising:
an array of transducer elements, the transducer elements of the array operable separably for transducing between acoustic and electrical energies, the array having a face at which the acoustic energies are transmitted and received and a back opposite the face;
a first backing adjacent the back of the array, the first backing being adjacent a first sub-set of the elements; and
a second backing adjacent the back of the array, the second backing being adjacent a second sub-set of the elements, the second sub-set different from the first sub-set;
wherein the first backing comprises hard backing material, the hard backing material having an acoustic impedance greater than a transducer layer of the transducer elements, and the second backing comprises soft backing material, the soft backing material having an acoustic impedance less than the transducer layer.

2. The ultrasound transducer of claim 1 wherein the acoustic impedance of the hard backing material is at least 50 MRayl, and the acoustic impedance of the soft backing material is at most 10 MRayl.

3. The ultrasound transducer of claim 2 wherein the acoustic impedance of the hard backing material is at least 60 MRayl, and the acoustic impedance of the transducer layer is about 30 MRayl.

4. The ultrasound transducer of claim 1 wherein the hard backing material comprises a block of solid material, and the soft backing material comprises a composite material.

5. The ultrasound transducer of claim 1 wherein the hard backing material comprises a first layer adjacent to the transducer elements of the first sub-set and further comprising a second layer of additional soft backing material adjacent the first layer such that the first layer is between the second layer and the transducer layer.

6. The ultrasound transducer of claim 1 wherein the transducer elements of the first sub-set are exclusive to the first sub-set and the transducer elements of the second sub-set are exclusive to the second sub-set.

7. The ultrasound transducer of claim 1 wherein the first and second sub-sets comprise contiguous rows of the transducer elements.

8. The ultrasound transducer of claim 1 wherein the first and second sub-sets comprise a checkerboard distribution of the transducer elements.

9. The ultrasound transducer of claim 1 wherein the transducer elements of the first and second sub-sets comprise a same thickness and same number of transducer layers including the transducer layer.

10. The ultrasound transducer of claim 1 wherein the transducer elements of the first sub-set having the hard backing material are operable at a lower frequency with a ¼ wavelength resonance, and the transducer elements of the second sub-set having the soft backing material are operable at a higher frequency with a ½ wavelength resonance.

11. A method for multi-purpose use of a medical ultrasound transducer, the method comprising:
transmitting acoustic energy at a first frequency from a first plurality of first elements, the first elements having a ¼ wavelength resonance at the first frequency, the ¼ wavelength resonance promoted by a backing with an acoustic impedance greater than a transducer material of the first elements; and
transmitting acoustic energy at a second frequency from a second plurality of second elements, the second elements having a ½ resonance at the second frequency, the ½ resonance promoted by a backing with an acoustic impedance less than a transducer material of the second elements;
wherein the first and second elements are part of a same array.

12. The method of claim 11 wherein the backing with the acoustic impedance greater than the transducer material of the first elements is about ¼ wavelength thick, and wherein the transmitting at the first frequency comprises transmitting with at least 30% of the acoustic energy reflected from a boundary at the backing and the transducer material of the first elements and at the backing and another backing.

13. The method of claim 12 wherein transmitting at the first frequency comprises transmitting with at least 90% of acoustic energy impinging on the backing being reflected.

14. The method of claim 11 wherein transmitting at the first frequency comprises transmitting the acoustic energy for therapeutic ultrasound and wherein transmitting at the second frequency comprises transmitting the acoustic energy for imaging.

15. The method of claim 11 wherein transmitting at the first frequency comprises transmitting the acoustic energy for a pushing pulse of acoustic radiation force ultrasound and wherein transmitting at the second frequency comprises transmitting the acoustic energy for imaging.

16. An ultrasound transducer for multi-purpose use, the transducer comprising:
first elements configured for transmission at a first frequency by a first backing; and
second elements configured for transmission at a second frequency, higher than the first frequency, by a second backing, the second backing softer than the first backing;
wherein the first and second elements are adjacent each other in an array.

17. The ultrasound transducer of claim 16 wherein an acoustic impedance of the first backing is at least 50 MRayl, and an acoustic impedance of the second backing is at most 10 MRayl, and an acoustic impedance difference of the first and second elements is at least 50 MRayl.

18. The ultrasound transducer of claim 16 wherein the first backing comprises a block of solid material, and the second backing comprises a composite material.

19. The ultrasound transducer of claim 16 wherein the first backing comprises a plurality of layers having different acoustic impedances such that reflections are caused at the first frequency.

20. The ultrasound transducer of claim 16 wherein the first and second elements have a same thickness between an emitting face and the respective first and second backing, the first and second elements distributed within the array by rows, rings or in a checkerboard pattern.
